(19) United States
(12) Patent Application Publication
(10) Pub. No.: US 2007/0265209 A1
(43) Pub. Date: Nov. 15, 2007

(54) Cosmetic or pharmaceutical oil-in-water emulsion

(76) Inventors: Caroline Goget, Paris (FR); Ulrich Issberner, Ambler, PA (US); Gabriele Strauss, Duesseldorf (DE); Roland Spoerer, Klongtoey (TH); Albert Frisch, Edesheim (DE)

Correspondence Address: COGNIS CORPORATION;PATENT DEPARTMENT, 300 BROOKSIDE AVENUE, AMBLER, PA, 19002, US

(21) Appl. No.: 11/638,972

(22) Filed: Dec. 14, 2006

(30) Foreign Application Priority Data
Dec. 20, 2005 (DE) 10 2005 061 239.3

(51) Int. Cl. A61K 31/70; A61K 31/20

(52) U.S. Cl. 514/23; 514/558

(57) ABSTRACT

A cosmetic or pharmaceutical oil-in-water (o/w) emulsion including an oil phase and a water phase, where at least 40% by weight of the oil phase is a wax component or a mixture of wax components is provided. A cosmetic or pharmaceutical oil-in-water (o/w) emulsion including 15% to 35% by weight of an oil phase which contains 40% to 70% by weight of a mixture of wax components comprising at least one C₁₆₋₂₄ fatty alcohol and at least one wax ester of a C₁₂₋₂₄ fatty alcohol and a C₁₂₋₂₄ fatty acid; 0.5% to 5% by weight of a nonionic alk(en)yl oligoglycoside surfactant; and 50% to 80% by weight water is also provided.

### CROSS-REFERENCE TO RELATED APPLICATIONS

This application claims priority under 35 U.S.C. § 119 from German Patent Application No. 102005061239.3, filed Dec. 20, 2005, the entire disclosure of which is hereby incorporated by reference.

### FIELD OF THE INVENTION

This invention relates to oil-in-water (o/w) emulsions, of which the oil phase contains a high percentage of wax components, and to their use for skin care and skin protection in special occupational sectors.

### BACKGROUND INFORMATION

Modern products for occupational skin protection have to be adapted to the particular working medium. In the case of water-miscible cooling lubricants as the working medium/noxae, the skin protection products hitherto used have been based on water-in-oil (w/o) emulsions which, by virtue of their highly lipophilic character, afford a natural protection, particularly against water. However, the use of water-in-oil emulsions represents a major “cultural” hurdle for the very people working in these sectors (mostly men), because emulsions of this type are not immediately absorbed and leave behind an often greasy and sticky film.

The problem addressed by the present invention was to provide formulations which would be very thoroughly and quickly absorbed into the skin, would have good skin care properties, would not leave a sticky film behind and would afford effective protection against water-containing noxae, more particularly cooling lubricants.

### SUMMARY OF THE INVENTION

Briefly described, according to an aspect of the invention, a cosmetic or pharmaceutical oil-in-water (o/w) emulsion including an oil phase and a water phase, where at least 40% by weight of the oil phase is a wax component or a mixture of wax components is provided.

According to another aspect of the invention, a cosmetic or pharmaceutical oil-in-water (o/w) emulsion including 15% to 35% by weight of an oil phase which contains 40% to 70% by weight of a mixture of wax components comprising at least one C₁₆₋₂₄ fatty alcohol and at least one wax ester of a C₁₂₋₂₄ fatty alcohol and a C₁₂₋₂₄ fatty acid; 0.5% to 5% by weight of a nonionic alk(en)yl oligoglycoside surfactant; and 50% to 80% by weight water is also provided.

### DETAILED DESCRIPTION OF THE INVENTION

It has now surprisingly been found that this problem can be solved by o/w emulsions with a relatively high percentage wax content of which the outer phase is water. Despite their high wax content, the o/w emulsions are very stable and are readily absorbed into the skin. In addition, various studies have shown that they afford excellent protection against various cooling lubricants, more particularly water-miscible cooling lubricants. Their protective effect is at least comparable with that of conventional w/o emulsions.

Accordingly, the present invention relates to cosmetic or pharmaceutical o/w emulsions comprising an oil phase and a water phase, at least 40% by weight of the oil phase being a wax component or a mixture of wax components. In a preferred embodiment of the invention, the wax component or mixture of wax components makes up 50 to 70% by weight and preferably 55 to 70% by weight of the oil phase. The melting point of at least one wax component is preferably at least 30° C., more particularly in the range from 40 to 80° C. and, in a most particularly preferred embodiment, in the range from 40 to 70° C.

The present invention also relates to the use of the compositions according to the invention for the skin care and skin protection of people coming into contact with cooling lubricants, more particularly water-miscible cooling lubricants.

Wax Components

Waxes are normally understood to be any natural or synthetic substances and mixtures having the following properties: they have a solid to brittle hard consistency, are coarsely to finely crystalline, transparent to opaque and melt above 30° C. without decomposing. Even slightly above their melting point, they are low in viscosity and non-stringing and are very temperature-dependent in their consistency and solubility. A wax component or mixture of wax components which melt(s) at 30° C. or higher may be used for the purposes of the present invention. They are present in the compositions according to the invention in a total quantity of at least 40% by weight, based on the oil phase. Compositions of which the oil phase contains 50 to 70% by weight waxes are particularly suitable for the purposes of the invention.

Fats and fat-like substances with a wax-like consistency may also be used as waxes in accordance with the invention. These include inter alia fats (triglycerides), mono- and diglycerides, natural and synthetic waxes, fatty and wax alcohols, fatty acids, esters of fatty alcohols and fatty acids and fatty acid amides, paraffins or Vaseline or mixtures of these substances. It is preferred to use a mixture of wax components having different melting points, so that a “melt cascade” is developed on the skin. A lasting skin care effect is thus guaranteed and the formulation is found to be particularly pleasant. The percentage content of the relatively high-melting waxes (40 to 80° C.) is at least 10% by weight and, more particularly, at least 20% by weight, based on the total quantity of wax components.

Fats in the context of the invention are understood to be triacylglycerols, i.e. the triple esters of fatty acids with glycerol. They preferably contain saturated, unbranched and unsubstituted fatty acid components. They may also be mixed esters, i.e. triple esters of glycerol with various fatty acids. So-called hardened fats and oils, such as hardened kernel and fruit oils, obtained by partial hydrogenation may be used in accordance with the invention and are particularly suitable as consistency factors. Vegetable hardened fats and oils, for example hardened castor oil, peanut oil, soybean oil, colza oil, rapeseed oil, cottonseed oil, soybean oil, sunflower oil, palm oil, palm kernel oil, linseed oil, almond oil, corn oil, olive oil, sesame oil, cocoa butter and coconut fat, are preferred and include, for example, the products Cegesoft® SBE (shea butter) and Cegesoft® SH (shorea butter) marketed by Cognis Deutschland GmbH & Co. KG.

Suitable fats are inter alia the triple esters of glycerol with C₁₂₋₆₀ fatty acids and in particular C₁₂₋₃₆ fatty acids. These include hydrogenated castor oil, a triple ester of glycerol and a hydroxystearic acid which is marketed, for example, under the name of Cutina® HR. Glycerol tristearate, glycerol tribehenate (for example Syncrowax® HRC), glycerol tripalmitate or the triglyceride mixtures known under the name of Syncrowax® HGLC.

Suitable wax components are, in particular, mono- and diglycerides and mixtures of these partial glycerides. The glyceride mixtures usable in accordance with the invention include the products Novata® AB and Novata® B (mixture of C₁₂₋₁₈ mono-, di- and triglycerides) and Cutina® MD or Cutina® GMS (glyceryl stearate) marketed by Cognis Deutschland GmbH & Co. KG.

Mixed esters and mixtures of mono-, di- and triglycerides are particularly suitable for the purposes of the invention because they have a relatively low tendency towards crystallization and thus improve the performance of the composition according to the invention.

Wax components preferred for the purposes of the invention include fatty alcohols, for example the C₁₂₋₅₀ fatty alcohols, more particularly C₁₆₋₂₄ fatty alcohols obtained from natural fats, oils and waxes such as, for example, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. According to the invention, saturated, unbranched fatty alcohols are preferred. Other suitable fatty alcohols are the fatty alcohol cuts obtained in the reduction of naturally occurring fats and oils such as, for example, bovine tallow, peanut oil, colza oil, cottonseed oil, soybean oil, sunflower oil, palm kernel oil, linseed oil, castor oil, corn oil, rapeseed oil, sesame oil, cocoa butter and coconut oil. However, synthetic alcohols, for example the linear, even-numbered fatty alcohols from Ziegler's synthesis (Alfols®) or the partly branched alcohols from the oxosynthesis (Dobanols®) may also be used. The C₁₄₋₁₈ fatty alcohols marketed for example by Cognis Deutschland GmbH & Co. KG under the name of Lanette® 16 (C₁₋₆ alcohol), Lanette® 14 (C₁₋₄ alcohol), Lanette® 0 (C_{16/18} alcohol) and Lanette® 22 (C_{18/22} alcohol) are particularly suitable for the purposes of the invention. Fatty alcohols give the compositions a dryer feeling on the skin and are therefore preferably used.

C₁₄₋₄₀ fatty acids or mixtures thereof may be used as additional wax components. These include, for example, myristic, pentadecanoic, palmitic, margaric, stearic, nonadecanoic, arachic, behenic, lignoceric, cerotic, melissic, erucic and elaeostearic acid and substituted fatty acids such as, for example, 12-hydroxystearic acid, and the amides or monoethanolamides of the fatty acids. This list is meant to be purely exemplary without any limiting character.

Other wax components suitable for use in accordance with the present invention are, for example, natural vegetable waxes, such as candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. According to the invention, it can be of advantage to use hydrogenated or hardened waxes. Natural waxes usable in accordance with the invention also include the mineral waxes, such as ceresine and ozocerite for example, or the petrochemical waxes, for example petrolatum, paraffin waxes and microwaxes. Other suitable wax components are chemically modified waxes, more particularly the hard waxes such as, for example, montan ester waxes, sasol waxes and hydrogenated jojoba waxes. Synthetic waxes usable in accordance with the invention include, for example, wax-like polyalkylene waxes and polyethylene glycol waxes. Vegetable waxes are preferred for the purposes of the invention. Paraffins and Vaseline may also be used as wax components.

The wax component may also be selected from the group of wax esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols, from the group of polyols esterified with carboxylic acids and also from the group of lactides of long-chain hydroxycarboxylic acids. Examples of such esters include C₁₆₋₄₀ alkyl stearates, C₂₀₋₄₀ alkyl stearates (for example Kesterwachs® K82H), C₂₀₋₄₀ dialkyl esters of dimer acids, C₁₈₋₃₈ alkyl hydroxystearoyl stearates or C₂₀₋₄₀ alkyl erucates, esters of alkylcarboxylic acids with pentaerythritol, dipentaerythritol, tripentaerythritol and tetrapentaerythritol. Other suitable wax components are C₃₀₋₅₀ alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. According to the invention, it is preferred to use wax esters of linear, saturated C₁₂₋₂₄ fatty alcohols and linear, saturated C₁₂₋₂₄ fatty acids or hydroxyfatty acids.

In one preferred embodiment, the emulsions according to the invention contain a wax component or a mixture of wax components selected from the group of fatty alcohols, hydrogenated mono-, di- and triglycerides or a mixture thereof, fatty acids, natural vegetable waxes, animal waxes, chemically modified waxes, synthetic waxes or wax esters. In another preferred embodiment, a wax ester is used in combination with at least one other wax component, preferably a C₁₂₋₂₄ fatty alcohol, as the wax component(s). A mixture of wax esters, fatty alcohol and partial glycerides, more particularly C₁₂₋₂₄ wax esters, C₁₂₋₂₄ fatty alcohols and C₁₂₋₂₄ partial glycerides, preferably in a quantity of 50 to 100% by weight, based on the total quantity of wax components, is most particularly preferred by virtue of the positive melt cascade effect.

Oil Components

The o/w emulsions according to the invention contain an oil phase which contains at least one oil component, but preferably a mixture of various oil components which are liquid at 20° C. The oil phase (including wax components, but not surfactants) makes up from 15 to 35% by weight, preferably from 20 to 30% by weight and more particularly from 25 to 30% by weight of the composition as a whole. Suitable oil components are, for example, the classes of compounds mentioned below. These include inter alia Guerbet alcohols based on fatty alcohols containing 6 to 18 and preferably 8 to 10 carbon atoms, liquid esters of linear or branched, saturated or unsaturated C₆₋₂₂ fatty acids with linear or branched, saturated or unsaturated C₆₋₂₂ fatty alcohols, more particularly 2-ethylhexanol. The following are mentioned by way of example: hexyl laurate, myristyl isostearate, myristyl oleate, cetyl isostearate, cetyl oleate, stearyl isostearate, stearyl oleate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl isostearate, behenyl oleate, erucyl isostearate, erucyl oleate. Other suitable esters are, for example, liquid esters of C₁₆₋₃₈ alkylhydroxy-carboxylic acids with linear or branched, saturated or unsaturated C₆₋₂₂ fatty alcohols, liquid esters of linear and/or branched, saturated or unsaturated fatty acids with polyhydric alcohols (for example propylene glycol, dimer diol or trimer triol) and/or Guerbet alcohols, triglycerides or triglyceride mixtures, mono-, di- and triglyceride mixtures, esters of C₆₋₂₂ fatty alcohols and/or Guerbet alcohols with aromatic carboxylic acids, more particularly benzoic acid, esters of C₂₋₁₂ dicarboxylic acids with linear or branched, saturated or unsaturated alcohols containing 1 to 22 carbon atoms or polyols containing 2 to 10 carbon atoms and 2 to 6 hydroxyl groups, vegetable oils, branched primary alcohols, substituted cyclohexanes, linear dialkyl carbonates, Guerbet carbonates based on fatty alcohols containing 6 to 18 and preferably 8 to 10 carbon atoms, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group such as, for example, Di-n-octyl Ether (Cetiol® OE) or ring opening products of epoxidized fatty acid esters with polyols, hydrocarbons, such as paraffin or mineral oils, silicone oils and oligo- or poly-α-olefins.

Emulsions which are characterized in that the oil phase contains at least one oil component selected from the group of dialkyl carbonates, triglycerides, dialkylethers, C₁₂₋₄₀ hydrocarbons or a mixture of these substances represent a preferred embodiment of the invention.

The dialkyl carbonates may be symmetrical or nonsymmetrical, branched or unbranched, saturated or unsaturated and may be produced by transesterification reactions known from the prior art. The dialkyl carbonates may be symmetrical or non-symmetrical, branched or unbranched, saturated or unsaturated. Dialkyl carbonates with C₆₋₂₄ alkyl chains, more especially di-n-octyl carbonate or di-(2-ethylhexyl)-carbonate or a mixture of these substances, are particularly suitable for the purposes of the invention. Of these, di-n-octyl carbonate is preferred.

The hydrocarbons suitable for use in accordance with the invention have a chain length of 8 to 40 carbon atoms. They may be branched or unbranched, saturated or unsaturated. Of these, branched, saturated C₈₋₄₀ alkanes are preferred. Both pure substances and mixtures may be used. The mixtures are normally mixtures of different isomeric compounds. Compositions containing C₁₀₋₃₀, preferably C₁₂₋₂₀ and, more particularly, C₁₆₋₂₀ alkanes are particularly suitable and, of these, a mixture of alkanes containing at least 10% by weight branched alkanes, based on the total quantity of alkanes, is particularly preferred. The alkanes are preferably branched, saturated alkanes. Mixtures of alkanes containing more than 1% by weight 5,8-diethyl dodecane and/or more than 1% by weight didecene are particularly suitable.

Surface-Active Substances

Another preferred embodiment of the composition according to the invention additionally contains at least one surfactant from the group of nonionic and/or anionic surfactants. According to the invention, partial glycerides, glycerides and fatty alcohols are not included among the surfactants, but belong to the group of waxes. The content of surfactant is determined by the type of formulation, but does not normally exceed 10% by weight. The preferred content is from 0.5 to 10% by weight, preferably from 0.5 to 5% by weight and more particularly from 1 to 3% by weight, based on the composition as a whole.

Typical examples of nonionic surfactants are fatty alcohol polyglycol ethers, polyglycerol esters, alkylphenol polyglycol ethers, fatty acid polyglycol esters, fatty acid amide polyglycol ethers, fatty amine polyglycol ethers, alkoxylated mono/di/triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. In one particularly preferred embodiment, the nonionic surfactant is selected from the group of alk(en)yl oligoglycosides.

Alk(en)yl oligoglycosides are known nonionic surfactants which correspond to formula (I):
R¹O—[G]ₚ (I)
where R¹ is an alk(en)yl group, G is a sugar unit containing 5 or 6 carbon atoms and p is a number of 1 to 10. They may be obtained by the relevant methods of preparative organic chemistry. EP 0301298 A1 and WO 90/03977 A are cited here as representative of the literature abundantly available on the subject.

The alk(en)yl oligoglycosides may be derived from aldoses or ketoses containing 5 or 6 carbon atoms, preferably glucose. Accordingly, the preferred alk(en)yl oligoglycosides are alk(en)yl oligoglucosides. The index p in general formula (I) indicates the degree of oligomerization (DP), i.e. the distribution of mono- and oligoglycosides, and is a number of 1 to 10. Whereas p in a given compound must always be an integer and, above all, may assume a value of 1 to 6, the value p for a certain alk(en)yl oligoglycoside is an analytically determined calculated quantity which is generally a broken number. Alk(en)yl oligoglycosides having an average degree of oligomerization p of 1.1 to 3.0 are preferably used. Alk(en)yl oligoglycosides having a degree of oligomerization of less than 1.7 and, more particularly, between 1.2 and 1.4 are preferred from the applicational perspective. Alkyl oligoglucosides in which the substituent R¹ is derived from primary C₈₋₂₄, preferably C₁₂₋₂₄ and more particularly C₁₆₋₁₈ alcohols are preferably used in accordance with the invention. Technical mixtures of the alcohols may also be used. According to the invention, it is particularly preferred to use Emulgade® PL 68/50 (Cognis Deutschland GmbH & Co. KG), a mixture of cetearyl alcohol and cetearyl glucoside containing ca. 50% by weight cetearyl glucoside. The alk(en)yl oligoglycoside or mixture of alk(en)yl oligoglycosides is preferably used in quantities of 0.1 to 5.0% by weight, based on the composition as a whole.

Anionic, cationic and/or amphoteric or zwitterionic surfactants/emulsifiers or a mixture of these surfactants/emulsifiers may also be present as surfactants in the compositions according to the invention.

Typical examples of anionic surfactants are soaps, alkyl benzene-sulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Preferred anionic surfactants are those which are particularly mild and kind to the skin. These include, in particular, sulfosuccinates, acyl lactylates, acyl tartrates, acyl glutamates and acyl aspartates, protein fatty acid condensates (particularly wheat-based vegetable products) and alkyl(ether) phosphates. According to the invention, it is particularly preferred to use Amphisol® K (INCl: Sodium Cetyl Phosphate), preferably in quantities of 0.1 to 2.0% by weight and more particularly in quantities of 0.1 to 1.0%, based on the composition as a whole.

Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, amino-propionates, aminoglycinates, imidazolinium betaines and sulfobetaines. The surfactants mentioned are all known compounds. Information on their structure and production can be found in relevant synoptic works. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Another preferred composition is characterized in that it contains the following components:
(a) 15 to 35% by weight of an oil phase which contains 40 to 70% by weight of a mixture of wax components comprising at least C₁₋₂₄ fatty alcohols and the wax ester of C₁₂₋₂₄ fatty alcohols and C₁₂₋₂₄ fatty acids,
(b) 0.5 to 5% by weight of a nonionic surfactant selected from the group of alk(en)yl oligoglycosides,
(c) 50 to 80% by weight water and
(d) optionally other auxiliaries and additives.
   Other Optional Auxiliaries and Additives

Depending on their intended application, the cosmetic formulations contain a number of other auxiliaries and additives such as, for example, thickeners, superfatting agents, stabilizers, polymers, lecithins, phospholipids, biogenic agents, UV protection factors, antioxidants, deodorants, film formers, swelling agents, insect repellents, hydrotropes, solubilizers, preservatives, perfume oils, dyes, etc. which are listed by way of example in the following. The quantities in which the particular additives are used is determined by the intended use.

Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, polyvinyl alcohol, polyvinyl pyrrolidone and bentonites such as, for example, Bentone® GeIVS-5PC (Rheox). Electrolytes, such as sodium chloride and ammonium chloride, are generally not compatible with compositions according to the invention in relatively high concentrations.

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. Typical UV-A filters are, in particular, derivatives of benzoyl methane. The UV-A and UV-B filters may of course also be used in the form of mixtures, for example combinations of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene), and esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are often combined with water-soluble filters such as, for example, 2-phenylbenzimidazole-5-sulfonic acid and alkali metal, alkaline earth metal, ammonium, alkylammonium, alkanolammonium and glucammonium salts thereof. Besides the soluble substances mentioned, insoluble light-blocking pigments, i.e. finely dispersed metal oxides or salts, may also be used for this purpose. Examples of suitable metal oxides are, in particular, zinc oxide and titanium dioxide. Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin.

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Suitable insect repellents are, for example, N,N-diethyl-m-toluamide, pentane-1,2-diol or 3-(N-n-butyl-N-acetylamino)-propionic acid ethyl ester), which is marketed as Insect Repellent® 3535 by Merck KGaA, and Butylacetylaminopropionate.

In addition, hydrotropes, for example ethanol, isopropyl alcohol or polyols, may be used to improve flow behavior. Suitable polyols preferably contain 2 to 15 carbon atoms and at least two hydroxyl groups.

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Suitable perfume oils are mixtures of natural and synthetic perfumes. Natural perfumes include the extracts of blossoms, stems and leaves, fruits, fruit peel, roots, woods, herbs and grasses, needles and branches, resins and balsams. Animal raw materials, for example civet and beaver, and synthetic perfume compounds of the ester, ether, aldehyde, ketone, alcohol and hydrocarbon type, are also suitable.

### EXAMPLES

TABLE 1

| | Skin protection formulations | | | | |
|---|---|---|---|---|---|
| Trade name (INCI) | 1 | 2 | 3 | 4 | 5 |
| Emulgade ® PL 68/50 | 5 | 5 | 5 | 5 | 5 |
| (Cetearyl Glucoside and Cetearyl Alcohol) | | | | | |
| Amphisol ® K (Potassium Cetyl Phosphate) | | | | 0.3 | 0.3 |
| Cutina ® MD (Glyceryl Stearate) | | | | 2 | 2 |
| Cetiol ® CC (Dicaprylyl Carbonate) | 5 | 5 | 5 | 5 | 5 |
| Myritol ® 331 (Cocoglyceride) | 2 | 2 | 2 | 2 | 2 |
| Cetiol ® S (Diethylhexylcyclohexane) | | | | | |
| Lunacera ® M (Ozocerite) | | | | | |
| Vaseline, Petrolatum | | | | | |
| Cegesoft ® PS6 (Olus) | 3 | 3 | 3 | 2 | 2 |
| Cegesoft ® SBE (Butyrospermum Parkii) | 1 | 1 | | | |
| Cegesoft ® SH (Shorea Stenoptera) | 2 | 2 | 1 | 1 | 1 |
| Cetiol ® MM (Myristyl Myristate) | 5 | 5 | 5 | 5 | 5 |
| Novata ® B (mono-, di-, triglycerides, C12-C18) | 5 | 5 | 2.5 | 2.5 | 2.5 |
| Beeswax 8100, Cera alba | 2 | 2 | 1 | 6 | 6 |
| Water, deionized | 69.13 | 64.5 | 70 | 53.7 | 53.7 |
| Zincum ® N 29 (Zinc Stearate) | | | | | |
| Dry Flo Plus ® (Aluminium Starch Octenyl Succinate) | | | | | |
| Microwax, Ozocerite | | | | | |
| Copherol ® F 1300 (Tocopherol) | 0.2 | | | | |
| Solbrol ® ICG (Iodopropynyl butylcarbamate) | 0.05 | | | | |
| Neo Dragocid ® Liquid (Triethylene Glycol, | 0.6 | | | | |
| Imidazolidinyl Urea, Methylparaben, Propylparaben, | | | | | |
| Dehydracetic acid) | | | | | |
| Ascorbylpalmitate | 0.02 | | | | |
| Hydagen ® CMF (10%); (Chitosan Glycolate) | | | | 10 | |
| Hydagen ® HCMS-LA (Chitosan Lactate) | | | | | 10 |
| Glycerin | | 5 | 5 | 5 | 5 |
| Uniphen ® P 23 (Phenoxyethanol, Methyl-, Ethyl-, | | 0.5 | 0.5 | 0.5 | 0.5 |
| Propyl-, Butylparabens) | | | | | |

TABLE 2

| | Skin protection formulations | | | | | |
|---|---|---|---|---|---|---|
| Trade name (INCI) | 6 | 7 | 8 | 9 | 10 | 11 |
| Emulgade ® PL 68/50 (Cetearyl Glucoside and | 5 | 5 | 5 | 5 | 5 | 5 |
| Cetearyl Alcohol) | | | | | | |
| Amphisol ® K (Potassium Cetyl Phosphate) | 0.3 | 0.3 | 0.3 | 0.3 | 0.3 | 0.3 |
| Cutina ® MD (Glyceryl Stearate) | 2 | 2 | 2 | 2 | 2 | 2 |
| Cetiol ® CC (Dicaprylyl Carbonate) | 5 | 5 | 5 | 5 | 5 | 5 |
| Myritol ® 331 (Cocoglyceride) | | | | | | 2 |
| Cetiol ® S (Diethylhexylcyclohexane) | 3 | 3 | 3 | 3 | 3 | |
| Lunacera ® M (Ozocerite) | | | | | | |
| Vaseline, Petrolatum | 6 | 6 | 6 | 6 | 6 | |
| Cegesoft ® PS6 (Olus) | 2 | 2 | 2 | 2 | 2 | 2 |
| Cegesoft ® SBE (Butyrospermum Parkii) | | | | | | |
| Cegesoft ® SH (Shorea Stenoptera) | | | | | | 1 |
| Cetiol ® MM (Myristyl Myristate) | 5 | 5 | 5 | 5 | 5 | 5 |
| Novata ® B (mono-, di-, triglycerides, C12-C18) | | | | | | 2.5 |
| Beeswax 8100, Cera alba | | | | 2 | | 6 |
| Water, deionized | 71.7 | 69.7 | 69.7 | 69.7 | 69.7 | 64.2 |
| Zincum ® N 29 (Zinc Stearate) | | 2 | | | | |
| Dry Flo Plus ® (Aluminium Starch Octenyl | | | 2 | | | |
| Succinate) | | | | | | |
| Microwax, Ozocerite | | | | | 2 | |
| Copherol ® F 1300 (Tocopherol) | | | | | | |
| Solbrol ® ICG (Iodopropynyl butylcarbamate) | | | | | | |
| Neo Dragocid ® Liquid (Triethylene Glycol, | | | | | | |
| Imidazolidinyl Urea, Methylparaben, | | | | | | |
| Propylparaben, Dehydracetic acid) | | | | | | |
| Ascorbylpalmitate | | | | | | |
| Hydagen ® CMF (10%); (Chitosan Glycolate) | | | | | | |
| Hydagen ® HCMS-LA (Chitosan Lactate) | | | | | | |
| Glycerin | | | | | | 5 |
| Uniphen ® P 23 (Phenoxyethanol, Methyl-, | | | | | | |
| Ethyl-, Propyl-, Butylparabens) | | | | | | |

Testing of the Formulations According to the Invention in Comparison with Commercially Available Products

The formulations according to the invention were tested for their care and protective effect against water- or fat-soluble noxae in the standardized BUS skin care model (bovine udder test) on follicular and non-follicular skin: Förster Th., Pittermann W., Schmitt M., Kietzmann M. (1999), “Skin penetration properties of cosmetic formulations using a perfused bovine udder model”; J. Cosmet. Sci. 50, 147-157; Jackwerth B., Pittermann W., Kietzmann M. (1996): “Bovine udder skin model (BUS): Innovatives in-vitro-Modell zur Penetration, Resorption und Imitationswirkung kosmetischer Stoffe und Formulierungen (Innovative in vitro model for the penetration, absorption and imitation effect of cosmetic agents and formulations)”; Parfumerie und Kosmetik 77, 37-40; Kietzmann M.; Löscher W., Arens D., Maaβ P., Lubach D. (1993): “Perfused bovine udder as an in-vitro model of percutaneous drug absorption. Skin viability and percutaneous absorption of dexamethasone, benzoyl peroxide and etofenamate”; J. Pharm. Toxicol. Meth. 30, 75-84; Pittermann W., Hörner V., Förster Th., Kietzmann M. (1997): “Use of natural and artificial skin models in cosmetic research”; SÖFW-Journal 123, 666-670; Pittermann W., Jackwerth B., Schmitt M. (1997): “The isolated perfused bovine udder skin model: A new in-vitro model for the assessment of skin penetration and irritation”; Invitro Toxicology 10, 17-21.

SDS (sodium dodecyl sulfate), toluene and water-miscible cooling lubricants were used as noxae for this purpose. The formulations according to the invention were performance-tested in comparison with two commercially available products: Saniwip®, a commercially available stearate cream, for example Stokolan®, an o/w skin-care emulsion. Vaseline (DAB) or wool fat wax (DAB) was used as the standard with 100% protection (=positive controls).

The principle is a) testing the dermatological compatibility of the products, b) the irritation potential of the noxae and c) the effects of the noxae after pretreatment of the skin with the test substances (products) as a function of the exposure time (15, 60 and 300 minutes after application of the noxae). The protection potential after a contact time of 15 minutes, which lasts for up to 5 hours according to quality, is compared. The earlier the protective effect is developed, the less the noxae are able to penetrate the skin and induce cellular reactions.

The test design allows the comparative use of two test substances and one noxa under the same experimental conditions. In this way, it is possible to measure the influence of the qualitatively different pretreatment on damage by the noxa. The percentage reduction in the noxa damage is used as the evaluation scale.

Overall, two formulations and two commercial skin care or skin protection products were tested for their effectiveness against SDS and toluene (water- or fat-soluble noxae). Toluene and SDS cause biochemically detectable skin damage after an exposure time of 60 and 300 minutes. Sometimes not enough damage is done after 15 minutes, so that no protection potential can be detected in the formulations or products. Both noxae induced essentially cytotoxic damage. Generally, the skin protection potential against fat-soluble noxae, such as toluene, is not as well developed as against SDS.

Skin Protection Application

Follicular/nonfollicular skin: the exposure time of each individual product (formulations according to the invention and Saniwip® and Stokolan® for comparison) was 0.25 h (beginning of noxa application) (quantity applied: 2 g/100 cm²), after which the product-treated area was covered with the noxa (for example cloth soaked with toluene) for 0.25 h, the noxa was removed with the cloth after 0.25 h and the first skin biopsy (0.25 h) was taken. The second and third biopsies were taken 1.0 h and 5.0 h after the beginning of the noxa application. Number of tests/test substance: follicular skin (n=4 udders, 72 whole skin biopsies); nonfollicular skin (n=2 udders; 24 whole skin biopsies).

The results are set out in the following Tables. The first value in the Tables relates to follicular skin, the value in brackets to nonfollicular skin.

1. Formulation According to the Invention in Comparison with Saniwip® (Tables 3-6); Noxa=Toluene or SDS

TABLE 3
Protective effect against damage by toluene: follicular skin
(not follicular skin)

| Test substance | Exposure: 0.25 h | Exposure: 1.0 h | Exposure: 5.0 h |
|---|---|---|---|
| Example 11 | Not determined | 59% (70%) | 42% (9%) |
| Saniwip ® | Not determined | 52% (44%) | 33% (13%) |

Compared with the two positive controls (Vaseline and wool fat wax=100%), the protection afforded by Example 11 and Saniwip® amounts to ca. 59% and 42%, respectively.

TABLE 4
Protective effect against damage by toluene: follicular skin
(not follicular skin)

| Test substance | Exposure: 0.25 h | Exposure: 1.0 h | Exposure: 5.0 h |
|---|---|---|---|
| Example 4 | Not determined | 42% (75%) | 46% (58%) |
| Saniwip ® | Not determined | 9% (30%) | 43% (29%) |

Compared with the two positive controls (Vaseline and wool fat wax=100%), the protection afforded by Example 4 and Saniwip® amounts to ca. 99% and 44%, respectively.

TABLE 5
Protective effect against damage by SDS (10% by weight): follicular skin
(nonfollicular skin)

| Test substance | Exposure: 0.25 h | Exposure: 1.0 h | Exposure: 5.0 h |
|---|---|---|---|
| Example 11 | Not determined | 70% (80%) | 100% (57%) |
| Saniwip ® | Not determined | 65% (69%) | 54% (66%) |

Compared with a positive control (Vaseline: 100%), the protection afforded by Example 11 and Saniwip® amounts to ca. 103% and 100%, respectively.

TABLE 6
Protective effect against damage (=100%) by SDS (10% by weight):
follicular skin (nonfollicular skin)

| Test substance | Exposure: 0.25 h | Exposure: 1.0 h | Exposure: 5.0 h |
|---|---|---|---|
| Example 4 | 100% | 86% (100%) | 85% (81%) |
| | (not determined) | | |
| Saniwip ® | 43% | 46% (23%) | 40% (36%) |
| | (not determined) | | |

Compared with a positive control (Vaseline: 100%), the protection afforded by Example 4 and Saniwip® amounts to ca. 136% and 62%, respectively.

2. Formulation According to the Invention in Comparison with Stokolan® (Tables 7, 8); Noxa=Toluene or SDS

TABLE 7
Protective effect against damage (=100%) by SDS (10% by weight):
follicular skin (nonfollicular skin)

| Test substance | Exposure: 0.25 h | Exposure: 1.0 h | Exposure: 5.0 h |
|---|---|---|---|
| Example 5 | Not determined | 54% (80%) | 53% (66%) |
| | (70%) | | |
| Stokolan ® | Not determined | 37% (23%) | 41% (33%) |
| | (0%) | | |

Compared with a positive control (Vaseline: 100%), the protection afforded by Example 5 and Stokolan® amounts to ca. 100% and 44%, respectively.

TABLE 8
Protective effect against damage (=100%) by toluene: follicular skin
(nonfollicular skin)

| Test substance | Exposure: 0.25 h | Exposure: 1.0 h | Exposure: 5.0 h |
|---|---|---|---|
| Example 5 | Not determined | 50% (45%) | 55% (48%) |
| | (53%) | | |
| Stokolan ® | Not determined | 24% (0%) | 31% (23%) |
| | (0%) | | |

Compared with the two positive controls (Vaseline, wool wax salve=100%), the protection afforded by Example 5 and Stokolan® amounts to ca. 70% and 18%, respectively.

3. Results and Summary of that of the Tables 3 to 8

TABLE 9
Table 9. Protective effect against damage by toluene or SDS
Reduction in noxa damage <40%: inadequate; care effect, but no
protection
Reduction in noxa damage <50%: satisfactory; care effect; some protection
Reduction in noxa damage >50%: good
Reduction in noxa damage >60%: very good
Reduction in noxa damage >80%: comparable with positive control
(Vaseline, wool wax salve)

| Test substance | Exposure: 0.25 h | Exposure: 1.0 h | Exposure: 5.0 h |
|---|---|---|---|
| Toluene/Example 11 | Not determined | Good | Satisfactory (inadequate) |
| | | (very good) | |
| Toluene/Saniwip ® | Not determined | Good | Inadequate (inadequate) |
| | | (satisfactory) | |
| Toluene/Example 4 | Not determined | Satisfactory | Satisfactory (good) |
| | | (as positive control) | |
| Toluene/Saniwip ® | Not determined | Inadequate | Satisfactory |
| | | (inadequate) | (inadequate) |
| Toluene/Example 5 | Not determined | Good | Good |
| | | (satisfactory) | (satisfactory) |
| Toluene/Stokolan ® | Not determined | Inadequate | Inadequate |
| | | (inadequate) | (inadequate) |
| SDS/Example 11 | Not determined | Very good | As positive control |
| | | (as positive control) | (good) |
| SDS/Saniwip ® | Not determined | Very good | Good |
| | | (very good) | (very good) |
| SDS/Example 4 | Not determined | As positive control | As positive control |
| | | (as positive control) | (as positive control) |
| SDS/Saniwip ® | Not determined | Satisfactory | Satisfactory (inadequate) |
| | | (inadequate) | |
| SDS/Example 5 | Not determined | Good | Good |
| | | (as positive control) | (very good) |
| SDS/Stokolan ® | Not determined | Inadequate | Satisfactory (inadequate) |
| | | (inadequate) | |

In standardized skin protection tests with the BUS model (follicular/nonfollicular skin), the skin protection potentials of the formulations of Example 11, Example 4 and Example 5 against toluene (lipid-soluble) and SDS (10% active substance) were tested in comparison with Saniwip® and Stokolan®. The contact time of the protection products was 15 minutes while the exposure times were 0.25 h, 1.0 h and 5.0 h. The formulations of Example 11 and Example 5 proved to be the most effective. Their protection effect corresponded to ca. 66% of the skin protection performance of the positive controls Vaseline or wool fat wax. The formulation of Example 4 also performed well.

A poorer protection effect than that of the formulations mentioned was achieved by Saniwip®. No protection effect against skin damage by toluene was achieved by application of Stokolan®.

A protection effect against SDS-induced skin damage comparable with that of the positive controls was achieved by application of the formulations of Example 11 and Example 4. The protection effect of the formulation of Example 5 was only marginally weaker. The protection effect achieved by Saniwip® against the water-soluble noxa SDS was also poorer than that of the formulations according to the invention. No protection effect was achieved by application of Stokolan®.

What is claimed is:

1. A cosmetic or pharmaceutical oil-in-water (o/w) emulsion comprising an oil phase and a water phase, wherein at least 40% by weight of the oil phase is a wax component or a mixture of wax components.

2. The emulsion according to claim 1, wherein the wax component or mixture of wax components makes up 50% to 70% by weight of the oil phase.

3. The emulsion according to claim 1, wherein the wax component or mixture of wax components makes up 55% to 70% by weight of the oil phase.

4. The emulsion according to claim 1, wherein at least one wax component has a melting point of at least 30° C.

5. The emulsion according to claim 1, wherein at least one wax component has a melting point ranging from about 40° C. to about 80° C.

6. The emulsion according to claim 1, wherein the wax component or mixture of wax components is selected from the group consisting of fatty alcohols, hydrogenated mono-, di- and triglycerides and mixtures thereof, fatty acids, natural vegetable waxes, animal waxes, chemically modified waxes, synthetic waxes, and wax esters.

7. The emulsion according to claim 1, wherein the mixture of wax components includes a wax ester and at least one other wax component.

8. The emulsion according to claim 1, wherein the oil phase makes up 15% to 35% by weight of the composition as a whole.

9. The emulsion according to claim 1, wherein the oil phase makes up 20% to 30% by weight of the composition as a whole.

10. The emulsion according to claim 1, wherein the oil phase contains at least one oil component selected from the group consisting of dialkyl carbonates, triglycerides, dialkyl ethers, C₁₂₋₄₀ hydrocarbons, and mixtures thereof.

11. The emulsion according to claim 1, further comprising at least one surfactant selected from the group consisting of non-ionic surfactants, anionic surfactants, and mixtures thereof.

12. The emulsion according to claim 11, wherein the nonionic surfactants are alk(en)yl oligoglycosides.

13. A cosmetic or pharmaceutical oil-in-water (o/w) emulsion comprising:
(a) 15% to 35% by weight of an oil phase which contains 40% to 70% by weight of a mixture of wax components comprising at least one C₁₆₋₂₄ fatty alcohol and at least one wax ester of a C₁₂₋₂₄ fatty alcohol and a C₁₂₋₂₄ fatty acid;
(b) 0.5% to 5% by weight of a nonionic alk(en)yl oligoglycoside surfactant; and
(c) 50% to 80% by weight water.

14. The emulsion according to claim 13, further comprising an auxiliary and/or additive.

15. The emulsion according to claim 1, incorporated into a preparation for applying to the skin to protect the skin from cooling lubricants.

16. The emulsion according to claim 13, incorporated into a preparation for applying to the skin to protect the skin from cooling lubricants.
